(19) United States
(12) Patent Application Publication
(10) Pub. No.: US 2003/0194371 A1
(43) Pub. Date: Oct. 16, 2003

(54) (Ethylene)-(propylene) - triaminepentaacetic acid derivatives, process for their production, and their use for the production of pharmaceutical agents

(75) Inventors: Lutz Lehmann, Berlin (DE); Matthias Friebe, Berlin (DE); Christoph-Stephan Hilger, Berlin (DE); Ulrich Niedballa, Berlin (DE); Johannes Platzek, Berlin (DE); Bernd Raduchel, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/191,987

(22) Filed: Jul. 10, 2002

Related U.S. Application Data
(60) Provisional application No. 60/306,141, filed on Jul. 19, 2001.

(30) Foreign Application Priority Data
Jul. 10, 2001 (DE) 101 33 435.4

(51) Int. Cl. A61K 51/00; A61K 49/00; C07F 5/00; C07H 21/04

(52) U.S. Cl. 424/1.11; 424/9.364; 534/11; 534/16; 530/405; 536/23.1; 558/12; 562/434; 560/330

(57) ABSTRACT

The invention relates to (ethylene)-(propylene)-triaminepentaacetic acid derivatives that are substituted on both the ethylene bridge and the propylene bridge, as well as conjugates of these compounds with biomolecules. The compounds and conjugates are suitable as agents for NMR diagnosis and radiodiagnosis as well as for radiotherapy.

This application claims the benefit of U.S. provisional application 60/306,141 which is incorporated by reference herein in its entirety, and U.S. provisional applications 60/300,479, 60/305,936, 60/328,108, and 60/335,106 and EPA 01117355.6 are all incorporated by reference herein in their entireties.

The invention relates to the subjects that are characterized in the claims, i.e., (ethylene)-(propylene)-triaminepentaacetic acid (EPTPA) derivatives, process for their production, and their use for the production of pharmaceutical agents for NMR diagnosis or radiodiagnosis or radiotherapy.

Nuclear magnetic resonance (NMR) is now an extensively used method of medical diagnosis that is exploited in in-vivo imaging with which bodily vessels and bodily tissue (including tumors) can be visualized via the measurement of the magnetic properties of the protons in the bodily water. To this end, e.g., contrast media are used that produce a contrast enhancement in the resulting images by influencing certain NMR parameters of the body protons (e.g., relaxation times T¹ and T²) or make these images readable only. Paramagnetic ions, such as, e.g., gadolinium-containing complexes (e.g., Magnevist^{(R)}), are primarily used because of the effect of the paramagnetic ions on the shortening of the relaxation times.

The use of radiopharmaceutical agents for diagnostic and therapeutic purposes has also been known for a long time in the area of biological and medical research. In particular, radiopharmaceutical agents are used to visualize certain structures, such as, for example, the skeleton, organs or tissue. The diagnostic application requires the use of such radioactive agents, which are concentrated after administration specifically in the structures in patients that are to be studied. These radioactive agents that accumulate locally can then be traced, plotted or scintigraphed by means of suitable detectors, such as, for example, scintillation cameras or other suitable imaging processes. The dispersion and relative intensity of the detected radioactive agent marks the site of a structure in which the radioactive agent is found, and the presence of anomalies in structures and functions, pathological changes, etc., can be visualized.

In a similar way, radiopharmaceutical agents can be used as therapeutic agents to irradiate certain pathological tissues or areas. Such treatment requires the production of radioactive therapeutic agents, which accumulate in certain structures, organs or tissues.

Both paramagnetic ions, such as, e.g.: Gd³⁺, Mn²⁺, Cr³⁺, Fe³⁺, and Cu²⁺ and many metallic radionuclides cannot be administered as solutions in free form since they are highly toxic. To make these ions suitable for an in-vivo application, they are generally complexed. For example, in EP-A-0 071 564, i.a., the meglumine salt of the gadolinium (III) complex of the diethylenetriaminepentaacetic acid (DTPA) is described as a contrast medium for the NMR tomography. A preparation that contains this complex was approved worldwide as the first NMR contrast medium under the name Magnevist^{(R)}. This contrast medium is dispersed extracellularly after intravenous administration and is excreted renally by glomerular secretion. A passage of intact cell membranes is virtually not observed. Magnevist^{(R)} is especially well suited for the visualization of pathological areas (e.g., inflammations, tumors).

The known contrast media and radiotherapeutic agents cannot be used satisfactorily for all applications, however. Many of these agents thus are dispersed into the entire extracellular space of the body. To increase the efficiency of these agents in in-vivo diagnosis and therapy, an attempt is made to increase their specificity and selectivity, for example on target cells or desired areas and structures of the body. An improvement of these properties is to be achieved by, for example, coupling metal complexes to biomolecules according to the “Drug-Targeting” principle. Biomolecules that can be considered include antibodies, their fragments, hormones, growth factors and substrates of receptors and enzymes (DE 195 36 780 A1).

In recent years, the need for diagnostic agents and therapeutic agents that accumulate specifically in diseased tissues has increased. In the coupling of complexing agents to selectively accumulating substances, it is often observed, however, that the complexing properties of the complexing agents deteriorate, so that a weakening of the complex stability can occur. In this respect, problems can arise if a physiologically relevant proportion of the toxic metal ions is released from the conjugate in vivo. In addition, a reduction of the specificity of the biomolecules by the conjugate formation in the chelating agents can result.

DTPA derivatives and their chelates with radioactive metal isotopes are disclosed in U.S. Pat. No. 5,248,764. The target specificity of these derivatives is achieved by coupling the DTPA via a carbonyl radical to a peptide. In this respect, this carbonyl radical for the complexing of the metal ion is lost, however, so that there is danger of an easier release of the toxic metal ion.

DTPA derivatives with a reactive side group, which is bonded to the methyl-carbon atom of a carboxymethyl side chain, are disclosed in EP-A-0 297 307. This has the advantage that none of the complex binding sites is blocked by the reactive side chain, with whose help the derivative can be coupled to, for example, a biomolecule. On the other hand, the reactive side chain in this position can exert an undesirable steric influence on the complexing and thus the complexing constants.

Other DTPA derivatives, which have, for example, a reactive benzyl group on an ethylene bridge and whose second ethylene bridge is also substituted, are disclosed in U.S. Pat. No. 4,831,175 and U.S. Pat. No. 5,124,471.

The chelating agent (ethylene)-(propylene)-triaminepentaacetic acid (EPTPA) was already described in DE 29 18 842 A1 for complexing heavy metal ions such as iron and manganese when bleaching wood pulp that can be used in the production of paper, where it is to facilitate the removal of such ions from the aqueous system that contains the wood pulp.

The use of the gadolinium (III) complex of EPTPA as an MRI contrast medium was described by Yun-Ming Wang et al. in J. Chem. Soc., Dalton Trans., 1998, 4113-4118. Moreover, this article discloses to one skilled in the art, surprisingly enough, that the gadolinium (III)-EPTA complex has a stability constant that is comparable to the gadolinium (III)-DTPA complex. This was especially surprising, therefore, since because of the ethylene bridges in DTPA, in each case two adjacent nitrogen atoms form with the latter a sterically ideal 5-ring when complexing the gadolinium ions, while a sterically less ideal 6-ring with the central gadolinium ion must be formed in EPTPA by the introduction of a propylene bridge.

In addition, it is desirable to make available agents for diagnosis and therapy that have as a high a target specificity as possible and that have as high an in-vivo stability as possible for the metal ions that are toxic in most cases.

An object of the invention was therefore to make available new agents for NMR diagnosis and radiodiagnosis as well as radiotherapy that do not exhibit the above-mentioned drawbacks and have in particular high in-vivo stability, good compatibility and primarily organ-specific properties. On the one hand, the retention in the organs that are to be examined is to be sufficient to obtain with a small dosage the number of images that are necessary for an unambiguous diagnosis, but, on the other hand, an excretion of the metals from the body that is as quick as possible and that is to a large extent complete is then to be ensured. The NMR contrast media also are to show high proton relaxivity and thus allow a reduction of the dose in the case of an increase in signal intensity.

It has now been found that this problem is solved by conjugates that consist of biomolecules with (ethylene)-(propylene/butylene)-triaminepentaacetic acid derivatives, whose ethylene bridge is substituted with a reactive benzyl group, and whose propylene bridge or butylene bridge has additional substituents. The invention thus relates to compounds of general formula I

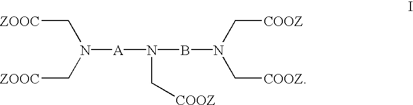

in which

Z stands for a hydrogen atom or a metal ion equivalent,

A stands for a radical of formula

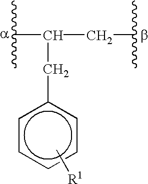

in which positions α and β that are characterized by

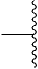

are bonded to any of the adjacent nitrogen atoms, R¹ is a nitro group or a group that can enter into a reaction with a biomolecule, and

B stands for a radical of formula

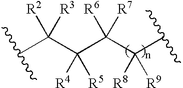

in which n is 0 or 1, and R², R³, R⁴, R⁵, R⁶, R⁷, R⁸ and R⁹, independently of one another, are selected from a hydrogen atom, a straight-chain or branched, saturated or unsaturated C₁₋₆ alkyl group, which optionally can be substituted with 1 or 2 hydroxy groups and/or can contain 1 or 2 oxygen atoms, and an aralkyl group, whose aryl radical optionally can be substituted with an alkyl or alkoxy group, whereby two of radicals R², R³, R⁴, R⁵, R⁶, R⁷, R⁸ and R⁹ can be part of a 5- or 6-membered ring, provided that at least one and at most four of radicals R², R³, R⁴, R⁵, R⁶, R⁷, R⁸, and R⁹ are not hydrogen atoms, as well as salts thereof, preferably with inorganic or organic bases.

Because of the reactive benzyl radical, the compounds according to the invention are suitable for the formation of conjugates with biomolecules, so that organ-specific properties are easily imparted to them, and the latter can be varied simply. Despite the substituted propylene or butylene bridge, metal complexes with the compounds according to the invention have high in-vivo stability. Moreover, the compounds according to the invention and their conjugates with biomolecules have good compatibility and good water solubility, so that they are suitable as pharmaceutical agents, especially for NMR diagnosis and radiodiagnosis as well as radiotherapy. The relaxivity of the complexes according to the invention is surprisingly high, so that the complexes, if they contain a paramagnetic ion, are especially well suited for NMR diagnosis.

In the compounds of Formula I according to the invention, A stands for a radical of formula

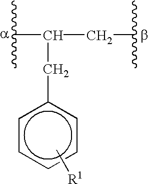

This radical can be bonded in positions α and β that are characterized by

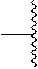

to any of the adjacent nitrogen atoms, i.e., the benzyl substituent of this radical can be adjacent to one of the two nitrogen atoms to which radical A is bonded. Radical A, however, is preferably bonded via position α to the (ZOOC—CH₂)₂—N radical, such that the benzyl substituent is adjacent to this radical.

The phenylene group of the benzyl substituent of radical A is substituted with a nitro group or a group that can enter into a reaction with a biomolecule. This substituent R¹ is preferably in meta- or para-position, in particular bonded to the phenylene groups in para-position. The compounds of formula I, in which R¹ is a nitrogen group, are especially well suited as intermediate compounds for the production of the compounds of formula I, in which R¹ is a group that can enter into a reaction with a biomolecule.

Suitable groups that can enter into a reaction with a biomolecule are, for example, amino (—NH₂), isocyanate (—NCO), isothiocyanate (—NCS), hydrazine (—NHNH₂), semicarbazide (—NHCONHNH₂), thiosemicarbazide (—NHCSNHNH₂), chloroacetamide (—NHCOCH₂Cl), bromoacetamide (—NHCOCH₂Br), iodoacetamide (—NHCOCH₂l), acylamino, such as, for example, acetylamino (—NHCOCH₃), maleimide, maleimidacylamino, such as, for example, 3-(2,5-dioxo-2,5-dihydro-pyrrol-1-yl)-propionylamino, activated esters, such as, for example,

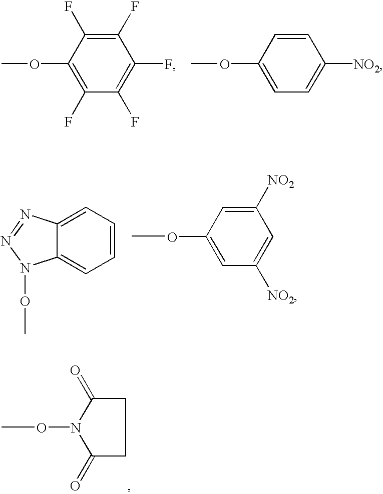

mixed anhydrides, azide, hydroxide, sulfonyl chloride and carbodiimide.

In formula I, B stands for a radical of formula

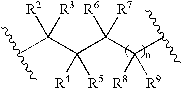

Hereinafter, n can be either 0 or 1, whereby compounds in which n=0 are preferred. The substituents R², R³, R⁴, R⁵, R⁶, R⁷, R⁸ and R⁹ are, independently of one another, selected from a hydrogen atom, a straight-chain or branched, saturated or unsaturated C₁₋₅ alkyl group, which optionally can be substituted with 1 or 2 hydroxy groups and/or can contain 1 or 2 oxygen atoms, and an aralkyl group, whose aryl radical optionally can be substituted with an alkyl or alkoxy group. In this connection, at least one and at most four of these radicals must not be hydrogen atoms, so that the propylene bridge or butylene bridge B in the compounds of formula I according to the invention carries at least one and at most four substituents.

The substituent or the substituents of bridge B can be a C₁₋₆ alkyl group, which optionally can be substituted with 1 or 2 hydroxy groups and/or can contain 1 or 2 oxygen atoms. Preferably the C₁₋₆ alkyl group is a methyl, ethyl, n-propyl, iso-propyl, n-butyl, iso-butyl or tert-butyl group. At the same time or alternately, one or more substituents of bridge B can be an aralkyl group, whereby aryl-C₁₋₆ alkyl groups and especially benzyl are preferred. The aryl radical of these aralkyl groups can be substituted preferably in para-position with an alkyl or alkoxy group. Preferably this alkyl group is a C₁₋₆ alkyl group, such as especially methyl or ethyl, and this alkoxy group is a C₁₋₆ alkoxy group, such as especially methoxy or ethoxy.

Bridge B is preferably substituted with 1 or 2 methyl, ethyl or benzyl groups.

Also preferred are those compounds according to the invention in which R², R³, R⁴, R⁵, R⁶, R⁷, R8 and R⁹ are selected, so that B is symmetrical.

In addition or as an alternative, two of radicals R², R³, R⁴, R⁵, R⁶, R⁷, R⁸ and R⁹ can be part of a 5- or 6-membered ring. The number of ring members of such a ring include the carbon atoms, to which the ring-forming radicals R², R³, R⁴, R⁵, R⁶, R⁷, R⁸ and R⁹ are bonded, as well as carbon atoms of propylene bridge or butylene bridge B that are optionally found in between. The ring can be saturated or unsaturated; 5- and 6-membered saturated rings are preferred.

Preferred propylene or butylene bridges B are: —CH₂—CH₂—CH(CH₂-CH₃)—, —CH(CH₂—CH₃)—CH₂—CH₂—, —CH₂—C(CH₃)₂—CH₂—, —CH₂—CH(CH₃)—CH₂, —CH₂—CH(CH₂-phenyl)—CH₂—, —CH₂—CH(CH₃)—CH(CH₃)—CH₂—.

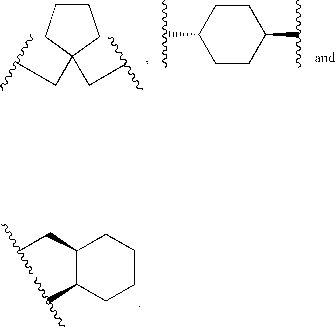

The compounds according to the invention contain at least one chirality center. Even if no distinction is made between the different enantiomers in the description and the claims, the above-mentioned compounds, if not otherwise indicated, always encompass both enantiomers and, in the presence of several stereo centers, also all possible diastereomers as well as mixtures thereof.

The compounds of formula I according to the invention are suitable for the production of conjugates with biomolecules. These conjugates have general formula II

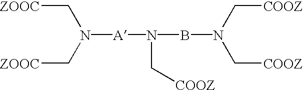

in which Z and B are defined above, and A′ stands for a radical of formula

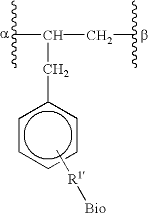

in which positions α and β that are characterized by

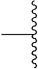

are bonded to any of the adjacent nitrogen atoms, and Bio stands for the radical of a biomolecule, which is bonded via radical R¹′ of a reactive group to the phenylene ring, as well as salts thereof, preferably with inorganic or organic bases. Radical R¹′ is preferably a radical R¹ as defined above after its reaction with a biomolecule.

“Biomolecule” is defined here as any molecule that either occurs naturally in, for example, the body or was produced synthetically with an analogous structure. Moreover, among the latter, those molecules are defined that can occur with a biological molecule that occurs, for example, in the body or a structure in interaction that occurs there, so that, for example, the conjugates can accumulate at certain desired spots of the body. “Body” is defined here as any plant or animal body, whereby animal and especially human bodies are preferred.

To form conjugates with the compounds according to the invention, the following biomolecules are especially suitable.

Biopolymers, proteins, such as proteins that have a biological function, HSA, BSA, etc., proteins and peptides that accumulate at certain spots in the organism (e.g., at receptors, cell membranes, ducts, etc.), peptides that can be cleaved by proteases, peptides with synthetic predetermined points of break (e.g., labile esters, amides, etc.), peptides that are cleaved by metalloproteases, peptides with photocleavable linkers, peptides with groups that can be cleaved with oxidative agents (oxydases), peptides with natural and unnatural amino acids, glycoproteins (glycopeptides), signal-proteins and antiviral proteins, synthetically modified biopolymers, such as biopolymers that are derivatized with linkers, modified metalloproteases and derivatized oxydase, etc., carbohydrates (mono- to polysaccharides), such as derivatized sugar, sugar that can be cleaved in the organism, cyclodextrins and derivatives thereof, amino sugar, chitosan, polysulfates and acetylneuraminic acid derivatives, antibodies, such as monoclonal antibodies, antibody fragments, polyclonal antibodies, miniborides, single chains (also those fragments that are linked by linkers to multiple fragments), red blood cells and other blood components, cancer markers (e.g., CAA) and cell-adhesion substances (e.g., Lewis X and Anti-Lewis X derivatives), DNA and RNA fragments, such as derivatized DNAs and RNAs (e.g., those that were found by the SELEX process, synthetic RNA and DNA (also with unnatural bases), PNAs (Hoechst) and antisense, β-amino acids (Seebach), vector amines for transfer into the cell, biogenic amines, pharmaceutical agents, oncological preparations, synthetic polymers, which are directed to a biological target (e.g., receptor), steroids (natural and modified), prostaglandins, taxol and derivatives thereof, endothelins, alkaloids, folic acid and derivatives thereof, bioactive lipids, fats, fatty acid esters, synthetically modified mono-, di- and tri-glycerides, liposomes that are derivatized on the surface, micelles that consist of natural fatty acids or perfluoroalkyl compounds, porphyrins, texaphrines, expanded porphyrins, cytochromes, inhibitors, neuramidases, neuropeptides, immunomodulators, such as FK 506, CAPE and gliotoxin, endoglycosidases, substrates that are activated by enzymes, such as calmodolin kinase, casein-kinase II, glutathione-S-transferase, heparinase, matrix-metalloproteases, β-insulin-receptor-kinase, UDP-galactose, 4-epimerase, fucosidases, G-proteins, galactosidases, glycosidases, glycosyltransferases and xylosidases, antibiotics, vitamins and vitamin analogs, hormones, DNA-intercalators, nucleosides, nucleotides, lectins, vitamin B12, Lewis-X and related substances, psoralens, dienetriene antibiotics, carbacyclins, VEGF (vascular endothelial growth factor), somatostatin and derivatives thereof, biotin derivatives, antihormones, dendrimers and cascade polymers as well as their derivatives, tumor-specific proteins and synthetic agents, polymers that accumulate in acidic or basic areas of the body (pH-controlled dispersion), myoglobins, apomyoglobins, etc., neurotransmitter peptides, tumor necrosis factors, peptides that accumulate in inflamed tissues, blood-pool reagents, anion and cation-transporter proteins, polyesters (e.g., lactic acid), polyamides and polyphosphates.

Most of the above-mentioned biomolecules are commercially available from, for example, Merck, Aldrich, Sigma, Calibochem and Bachem.

In addition, all “plasma protein binding groups” or “target binding groups” that are disclosed in WO 96/23526 and WO 01/08712 can be used as biomolecules. The content of these two laid-open specifications is therefore integrated by reference to this description.

The compounds according to the invention are also suitable for conjugation on all molecules that are reacted with fluorescence dyes in the prior art to determine, for example, their location by epifluorescence microscopy within the cell. After the administration of the medication, the compounds with, in principle, any medications can also be conjugated to then track the transport within the organism, for example by the NMR technique. It is also possible that the conjugates from the compounds according to the invention and the biomolecules contain other additional molecules, which had been conjugated on the biomolecules. The term “biomolecule” in terms of this invention thus encompasses all molecules that occur in the biological systems and all molecules that are biocompatible.

The compounds of general formula I and conjugates thereof with biomolecules can be obtained, for example, by reaction of a compound of formula III

H₂N-A-NH—B—NH₂ III

whereby A and B are as defined above, with a compound of formula IV

Nu-CH₂—COOZ′ IV,

whereby Nu stands for a nucleofuge and Z′ stands for a hydrogen atom, a metal ion equivalent, preferably an alkali or alkaline-earth metal, such as especially sodium or potassium, or a protective group for carboxyl. The compound that is thus obtained can then be reacted with a biomolecule, whereby radical R¹, if it is nitro, first must be converted into a group that can enter into a reaction with a biomolecule. After that, and after the removal of optionally still present protective groups, and in a way that is known in the art, a reaction can be performed, if desired, with at least one metal oxide or metal salt to obtain the desired metal complexes. In the complexes that are thus obtained, still present acidic hydrogen atoms, if desired, can then optionally be completely or partially substituted by cations of inorganic and/or organic bases, amino acids or amino acid amides.

As a nucleofuge, the radicals that are advantageously used are:

Cl, I, Br, —OTs, —OMs and —O-triflate.

The reaction is performed in a mixture of water and organic solvents, such as: isopropanol, ethanol, methanol, butanol, dioxane, tetrahydrofuran, dimethylformamide, dimethylacetamide, formamide or dichloromethane. Preferred are ternary mixtures that consist of water, isopropanol and dichloromethane.

The reaction is performed in a temperature range of between −10° C. and 100° C., preferably between 0° C. and 30° C.

The neutralization of optionally still present free carboxy groups is carried out with the aid of inorganic bases (e.g., hydroxides, carbonates or bicarbonates) of, e.g., sodium, potassium, lithium, magnesium, or calcium and/or organic bases, such as, i.a., primary, secondary and tertiary amines, such as e.g., ethanolamine, morpholine, glucamine, N-methylglucamine and N,N-dimethylglucamine, as well as basic amino acids, such as, e.g., lysine, arginine, and ornithine or amides of originally neutral or acidic amino acids.

For the production of neutral complex compounds, for example, enough of the desired bases can be added in acidic complex salts in aqueous solution or suspension to ensure that the neutral point is reached. The solution that is obtained can then be evaporated to the dry state in a vacuum. It is often advantageous to precipitate the formed neutral salts by adding water-miscible solvents, such as, e.g., lower alcohols (methanol, ethanol, isopropanol and others), low ketones (acetone and others), polar ethers (tetrahydrofuran, dioxane, 1,2-dimethoxyethane and others) and to obtain crystallizates that are easily isolated and readily purified. It has proven especially advantageous to add the desired base as early as during the complexing of the reaction mixture and thus to save a process step.

The production of the compounds of formula I according to the invention is explained below in the example of a preferred compound, in which R¹ is —NO₂, and B is R—CH₂—CH₂—CH(—CH₂—CH₃)—, and Z means hydrogen. The production of the compound

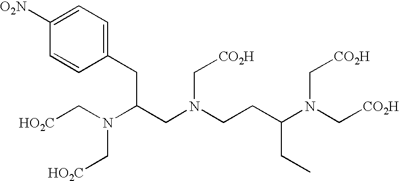

can be carried out from the t-butylesters 1

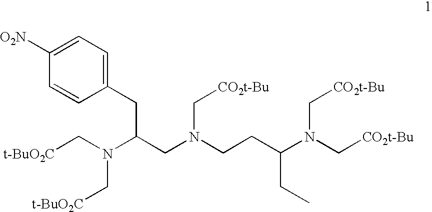

by acidic hydrolysis with trifluoroacetic acid. The compound of formula 1 can be obtained by alkylation of the amine of formula 2

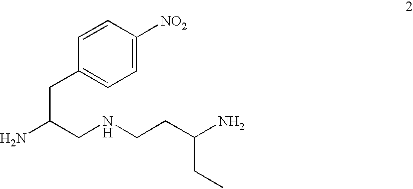

with bromoacetic acid-t-butyl ester. The ester can be obtained from Merck, Fluka or Aldrich.

Amine 2 can be obtained by hydrolysis with trifluoroacetic acid from desired compound 3

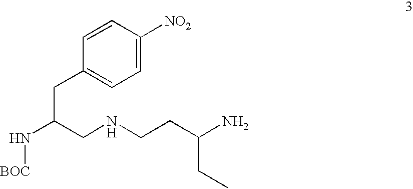

Compound 3 is available by alkylation of mesylate 4 with 1,3-diaminopentane 5 and chromatographic separation of the amino mixture.

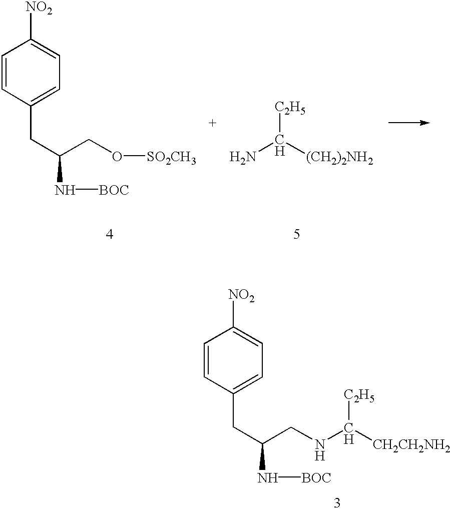

Amine 5 is commercially available (Aldrich, Fluka).

Mesylate 4 can be obtained by reaction of alcohol 6 with methanesulfonyl chloride in the presence of triethylamine.

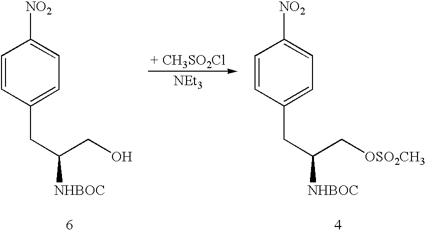

Alcohol 6 can be obtained by reduction of ester 7 with sodium borohydride in tetrahydrofuran/methanol (8:1):

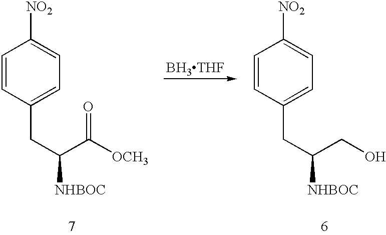

4-Nitrophenylalanine methyl ester 7 can be produced from corresponding acid 8 by esterification with methyl iodide in the presence of sodium bicarbonate in dimethylformamide:

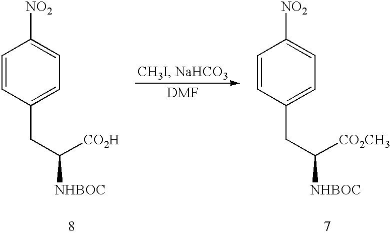

Acid 8 is commercially available (Aldrich, Fluka).

As an alternative, the procedure can also be such that component 3 is obtained by amide formation from phenylalanine 8 and 1-ethyl-1,3-propanediamine 5 and subsequent reduction of the amide bond. The chromatographic separation can be avoided, if the amine component is used as a 3-N—BOC derivative.

The α, ω-diamines that are required for the synthesis are available, for example, via the synthesis methods that are depicted grammatically below:

C-3 Diamines, Substituents at C-2

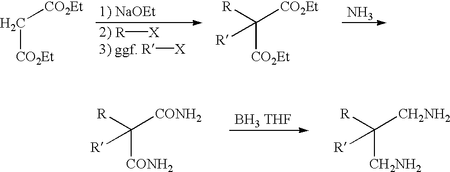

R: Alkyl, etc.

R¹: Hydrogen (then step 3 of the first stage is unnecessary) or R

X: Halogen

C-3 Diamines, Substituent at C-1

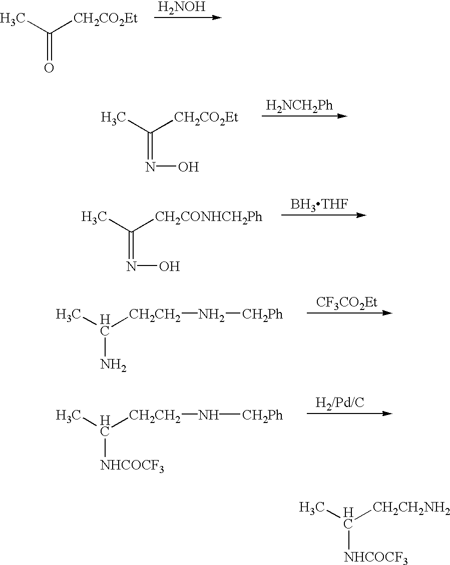

C4 Diamines, Substituent at C-4

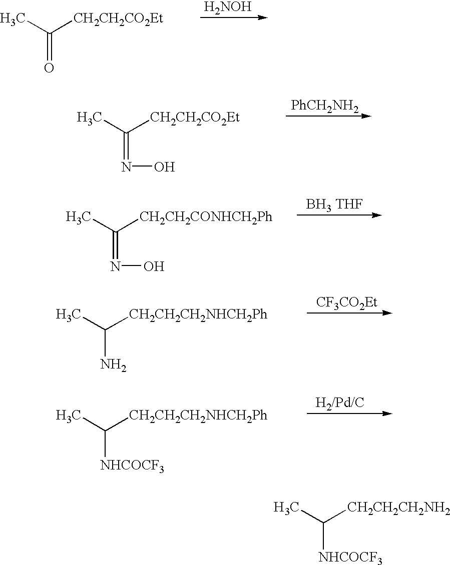

The conversion of the compounds of general formula I, in which R¹=NO₂, into compounds of general formula I, in which R¹ is not equal to nitro, is carried out according to known methods. The hydrogenation of the nitro group into the amino group is possible with, for example, 10% palladium on carbon (cf. EP 475 617; EP 173 629 and U.S. Pat. No. 5,087,696).

The amino group can be converted into the corresponding amide by reaction with nitrophenyl or hydroxysuccinimide esters. It can also be converted, however, by reaction with thiosphosgene in the isothiocyanate, which couples directly with amino groups to the thiourea. The isothiocyanate can also react with hydrazine to form thiosemicarbazide, which then is reacted specifically with the oxidized sugar molecules of an antibody to form thiosemicarbazide.

Analogously, amine is reacted with phosgene to form isocyanate, and the latter is reacted with hydrazine to form semicarbazone. The anilino group can also be acylated. If the reaction with the activated ester of the 4-maleimidobutyric acid (Fluka) is performed, a specific reagent that binds to —SH groups is obtained. The haloacetamides, which can be obtained by reaction of the anilines with haloactivated esters, also bind to —SH groups.

The amino group itself can also be used as a binding site for the carbonyl groups of oxidized sugar, if the partner molecule tolerates the conditions of reductive animation.

The production of complexes for the production of NMR diagnostic agents can be carried out in the way that was disclosed in Patents EP 71564, EP 130934 and DE-OS 34 01 052. To this end, the metal oxide or a metal salt (for example, chloride, nitrate, acetate, carbonate or sulfate) of the desired element in water and/or a lower alcohol (such as methanol, ethanol or isopropanol) is dissolved or suspended and reacted with the solution or suspension of the equivalent amount of the complexing agent according to the invention.

If the complexing agents are to be used for the production of radiodiagnostic or radiotherapeutic agents, the production of the complexes from the complexing agents can be carried out according to the methods described in “Radiotracers for Medical Applications,” Volume 1, CRC Press, Boca Raton, Fla.

It may be desirable to produce the complex only shortly before its use, especially if it is to be used as a radiopharmaceutical agent. The invention therefore also comprises a kit for the production of radiopharmaceutical agents encompassing a compound of formula I and a conjugate of formula II, in which Z is hydrogen, and a compound of a desired metal.

Subjects of the invention are also pharmaceutical agents that contain at least one physiologically compatible compound of general formula I or at least one physiologically compatible conjugate of general formula II, optionally with the additives that are commonly used in galenicals.

The production of the pharmaceutical agents according to the invention is carried out in a way that is known in the art, by the complex compounds—optionally with the addition of the additives that are commonly used in galenicals—being suspended or dissolved in aqueous medium, and then the suspension or solution optionally being sterilized. Suitable additives are, for example, physiologically harmless buffers (such as, e.g., tromoethamine), additions of complexing agents or weak complexes (such as, e.g., diethylenetriaminepentaacetic acid or the Ca complexes corresponding to the metal complexes according to the invention) or—if necessary—electrolytes, such as, e.g., sodium chloride or—if necessary—antioxidants, such as, e.g., ascorbic acid.

If suspensions or solutions of the agents according to the invention in water or physiological salt solution are desired for enteral administration or other purposes, they are mixed with one or more additive(s) that are commonly used in galenicals [e.g., methyl cellulose, lactose, mannitol] and/or surfactant(s) [e.g., lecithins, Tween^{(R)}, Myrj^{(R)}] and/or flavoring substance(s) for taste correction [e.g., ethereal oils].

In principle, it is also possible to produce the pharmaceutical agents according to the invention even without isolating the complex salts. In any case, special care must be taken to perform the chelation so that the salts and salt solutions according to the invention are virtually free of non-complexed metal ions that have a toxic effect.

This can be ensured, for example, with the aid of color indicators, such as xylenol orange, by control titrations during the production process. The invention therefore also relates to the process for the production of complex compounds and salts thereof. Purification of the isolated complex salt is a last safety measure.

The pharmaceutical agents according to the invention preferably contain 1 fmol-1.3 mol/l of the complex salt and are generally dosed in amounts of 0.0001-5 mmol/kg. They are intended for enteral and parenteral administration. The complex compounds according to the invention are used

1. For NMR diagnosis in the form of their complexes with the paramagnetic ions of the elements with atomic numbers 21-29, 42, 44 and 58-70. Suitable ions are, for example, the chromium (III), iron (II), cobalt (II), nickel (II), copper (II), praseodymium (III), neodymium (III), samarium (III) and ytterbium (III) ions. Because of their strong magnetic moment, the gadolinium (III), terbium (III), dysprosium (III), holmium (III), erbium (III), manganese (II) and iron (III) ions are especially preferred for NMR diagnosis.

2. For radiodiagnosis and radiotherapy in the form of their complexes with the radioisotopes of the elements with atomic numbers 26, 27, 29, 31, 32, 37-39, 43, 46, 47, 49, 61, 62, 64, 67, 70, 71, 75, 77, 82 and 83.

The agents according to the invention meet the many different requirements for suitability as contrast media for nuclear spin tomography. After oral or parenteral administration, they are thus extremely well suited for enhancing the information value of the image that is obtained with the aid of a nuclear spin tomograph by increasing the signal intensity. They also show the great effectiveness that is necessary to load the body with the minimum possible amounts of foreign substances, and the good compatibility that is necessary to maintain the non-invasive nature of the studies.

Good water-solubility and low osmolality of the agents according to the invention allow the production of highly concentrated solutions to keep the volume burden of the circulatory system within reasonable limits and to offset the dilution by bodily fluids, i.e., NMR diagnostic agents have to be 100 to 1000 times more water-soluble than for NMR spectroscopy. In addition, the agents according to the invention do not have only high stability in vitro, but also surprisingly high stability in vivo, such that a release or an exchange of the ions —that are inherently toxic—and that are not covalently bonded in the complexes takes place only extremely slowly within the time in which the new contrast media are completely excreted again.

In general, the agents according to the invention for use as NMR diagnostic agents are dosed in amounts of 0.0001-5 mmol/kg, preferably 0.005-0.5 mmol/kg. Details of use are discussed in, e.g., H. -J. Weinmann et al., Am. J. of Roentgenology 142, 619 (1984).

Low dosages (below 1 mg/kg of body weight) of organ-specific NMR diagnostic agents can be used, for example, for detecting tumors and myocardial infarction. Especially low dosages of the complexes according to the invention are suitable for use in radiotherapy and radiodiagnosis.

The complex compounds according to the invention can also be used advantageously as susceptibility reagents and as shift reagents for in-vivo NMR spectroscopy.

Because of their advantageous radioactive properties and the good stability of the complex compounds that are contained therein, the agents according to the invention are also suitable as radiodiagnostic agents and radiotherapeutic agents. Details of their use and dosage are described in, e.g., “Radiotracers for Medical Applications,” CRC Press, Boca Raton, Fla., 1983, as well as in Eur. J. Nucl. Med. 17 (1990), 346-364 and Chem. Rev. 93 (1993) 1137-1156.

For SPECT, complexes with the isotopes ¹¹¹In and ^{99m}Tc are suitable.

Another imaging method with radioisotopes is the positron-emission-tomography, which uses positron-emitting isotopes, such as, e.g., ⁴³Sc, ⁴⁴Sc, ⁵²Fe, ⁵⁵Co, ⁵⁸Ga, ⁶⁴Cu, ⁸⁶Y and ^{94m}Tc (Heiss W. D.; Phelps, M. E.; Positron Emission Tomography of Brain, Springer Verlag Berlin, Heidelberg, New York 1983).

The compounds according to the invention are also suitable, surprisingly enough, for differentiating malignant and benign tumors in areas without blood-brain barriers.

They are distinguished in that they are completely eliminated from the body and thus are well compatible.

Since the substances according to the invention accumulate in malignant tumors (no diffusion in healthy tissue, but high permeability of tumor vessels), they can also support the radiation therapy of malignant tumors. This is different from the corresponding diagnosis only by the amount and type of the isotope that is used. The purpose in this case is the destruction of tumor cells by high-energy short wave radiation with as small a range of action as possible. To this end, interactions of the metals that are contained in the complexes (such as, e.g., iron or gadolinium) are used with ionizing radiations (e.g., x rays) or with neutron rays. The local radiation dose at the site where the metal complex is found (e.g., in tumors) is significantly increased by these effects. To produce the same radiation dose in malignant tissue, the radiation exposure for healthy tissue can be considerably reduced when using such metal complexes and thus burdensome side effects for the patients can be avoided. The metal complex conjugates according to the invention are therefore also suitable as radiosensitizing substances in the radiation therapy of malignant tumors (e.g., use of the Mössbauer effects or in neutron capture therapy). Suitable β-emitting ions are, e.g., ⁴⁶Sc, ⁴⁷Sc, ⁴⁸Sc, ⁷²Ga, ⁷³Ga, ⁹⁰Y, ⁶⁷Cu, ¹⁰⁹Pd, ¹¹¹Ag, ¹⁴⁹Pm, ¹⁵³Sm, ¹⁶⁶Ho, ¹⁷⁷Lu, ¹⁸⁶Re, and ¹⁸⁸Re, whereby ⁹⁰Y, ¹⁷⁷Lu, ⁷²Ga, ¹⁵³Sm and ⁶⁷Cu are preferred. Suitably short half-lives that have α-emitting ions are, e.g., ²¹¹At, ²¹¹Bi, ²¹²Bi, ²¹³Bi and ²¹⁴Bi, whereby ²¹²Bi is preferred. A suitable photon- and electron-emitting ion is ¹⁵⁸Gd, which can be obtained from ¹⁵⁷Gd by neutron capture.

If the agent according to the invention is intended for use in the variant of radiation therapy that is proposed by R. L. Mills et al. [Nature Vol. 336, (1988), p. 787], the central ion must be derived from a Möβbauer isotope, such as, for example, ⁵⁷Fe or ¹⁶¹Eu.

In the in-vivo administration of the therapeutic agents according to the invention, the latter can be administered together with a suitable vehicle, such as, e.g., serum or physiological common salt solution, and together with another protein, such as, e.g., human serum albumin. In this case, the dosage is dependent on the type of cellular disorder, the metal ion that is used and the type of imaging method.

The therapeutic agents according to the invention are administered parenterally, preferably i.v.

Details of the applications of radiotherapeutic agents are discussed in, e.g., R. W. Kozak et al., TIBTEC, October 1988, 262 (see above Bioconjugate Chem. 12 (2001) 7-34).

In summary, it has been possible to synthesize new complexing agents, metal complexes and metal complex salts that open up new possibilities in diagnostic and therapeutic medicine.

The examples below are used for a more detailed explanation of the subject according to the invention.

The entire disclosures of all applications, patents and publications cited herein and of corresponding German application No. ______, filed Jul. 10, 2001 (Client Reference No. 52,165) are incorporated by reference herein.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLE 1

a) 2-tert-Butoxycarbonylamino-3-(4-nitro-phenyl)-propionic acid methyl ester

C₁₅H₂₀N₂O₆ (M=324.34)

A suspension that consists of 50 g (161.0 mmol) of Boc-NO₂-Phe (Bachem), 40.5 g (483 mmol) of sodium bicarbonate and 25.0 g (177 mmol) of methyl iodide in 600 ml of dimethylformamide was stirred for four days at room temperature. The suspension was suctioned off, and the solid was washed with dichloromethane. The filtrate was concentrated by evaporation in a rotary evaporator. The residue was taken up in water and extracted four times with ethyl acetate (150 ml each). The combined, organic phases were washed with 100 ml of 5% sodium thiosulfate solution, with 100 ml of 5% sodium bicarbonate solution, with 50 ml of saturated sodium chloride solution, with 100 ml of 10% citric acid and 50 ml of water. The organic phase was dried on sodium sulfate, filtered and concentrated by evaporation in a rotary evaporator. A raw yield of 46.5 9 (143.5 mmol), which corresponds to 89.4%, was produced.

| | | | | |
|---|---|---|---|---|
| Cld.: | C 55.55 | H 6.22 | N 8.64 | O 29.60 |
| Fnd.: | C 55.59 | H 6.23 | N 8.62 | O 29.63 |

b) (2-Hydroxy-1-(4-nitro-benzyl)-ethyl]-carbamic acid tert-butyl ester

C₁₄H₂₀N₂O₅ (M=296.32)

8.9 g (27.4 mmol) of 1a was dissolved in 70 ml of tetrahydrofuran. 2.0 g (51.2 mmol) of,sodium borohydride was added. 13 ml of methanol was slowly added in drops. The reaction solution was stirred overnight. The reaction solution was mixed with 2.8 ml of acetic acid and evaporated to the dry state. The residue was taken up in water and extracted with ethyl acetate. The combined organic phases were washed twice with saturated sodium chloride solution. The organic phase was dried on sodium sulfate, filtered off and concentrated by evaporation. Residual water with toluene was removed by azeotropic distillation in a rotary evaporator. The desired crude product was produced with a yield of 79.0% (6.4 g; 21.6 mmol).

| | | | | |
|---|---|---|---|---|
| Cld.: | C 56.75 | H 6.80 | N 9.45 | O 27.00 |
| Fnd.: | C 56.69 | H 6.75 | N 9.47 | O 27.07 |

c) Methanesulfonic acid 2-tert-butoxycarbonylamino-3-(4-nitro-phenyl)-propyl ester

C₁₅H₂₂N₂O₇S (M=374.41)

6.40 g (21.6 mmol) of 1b was dissolved in 5 ml of dichloromethane and 0.33 g (3.24 mmol) of triethylamine. The solution was cooled to −5° C. and slowly mixed with 0.27 g (2.38 mmol) of methanosulfonic acid chloride, which was diluted in some dichloromethane. The suspension was stirred for two hours and poured onto stirred ice water (about 50 ml). The phases were separated. The aqueous phase was extracted three times with dichloromethane (50 ml each). The combined organic phases were washed twice with dilute (5%) aqueous HCl solution, with water, with dilute (5%) sodium bicarbonate solution and with saturated sodium chloride solution. The organic phase was dried on sodium sulfate and evaporated to the dry state in a rotary evaporator. A yield of 98.9% of crude product was produced.

| | | | | | |
|---|---|---|---|---|---|
| Cld.: | C 48.12 | H 5.92 | N 7.48 | O 29.91 | S 8.56 |
| Fnd.: | C 48.21 | H 5.98 | N 7.43 | O 29.90 | S 8.57 |

d) [2-(3-Amino-pentylamino)-1-(4-nitro-benzyl)-ethyl]-carbamic acid tert-butyl ester

C₁₉H₃₂N₄O₄ (M=380.49)

10 g (26.71 mmol) of 1c was reacted with 27.85 g (267.1 mmol) of 1,3-diamino-pentane, 2.96 g (29.3 mmol) of triethylamine and 100 ml of tetrahydrofuran analogously to Example 3a. The product was produced with a yield of 50.6% (5.14 g; 13.51 mmol). In addition, it was possible to isolate 3.75 g (9.87 mmol) of [2-(3-amino-1-ethyl-propylamino)-1-(4-nitro-benzyl)-ethyl]-carbamic acid tert-butyl ester.

| | | | | |
|---|---|---|---|---|
| Cld.: | C 59.98 | H 8.84 | N 14.73 | O 16.82 |
| Fnd.: | C 59.90 | H 8.86 | N 14.75 | O 16.77 |

e) N′-[2-Amino-3-(4-nitro-phenyl)-propyl]-pentane-1,3-diamine

C₁₄H₂₄N₄O₂ (M=280.37)

3.65 g (9.59 mmol) of 1d was dissolved in 55 ml of dichloromethane and mixed with 16.24 g (142.46 mmol) of trifluoroacetic acid. The solution was stirred for 90 minutes at room temperature and concentrated by evaporation in a rotary evaporator. It was mixed twice with dichloromethane and concentrated by evaporation again in each case. The crude product was mixed with 100 ml of 5% ammonia solution. The product was freeze-dried. The operating step described most recently was repeated. The desired product was produced with 2.39 g (8.54 mmol, 89%).

| | | | | |
|---|---|---|---|---|
| Cld.: | C 59.98 | H 8.63 | N 19.98 | O 11.41 |
| Fnd.: | C 59.90 | H 8.62 | N 19.92 | O 11.44 |

f) {[2-{[3-(Bis-tert-butoxycarbonylmethyl-amino)-pentyl]-tert-butoxycarbonylmethyl-amino}-1-(4-nitro-benzyl)-ethyl]-tert-butoxycarbonylmethyl-amino}-acetic acid tert-butyl ester

C₄₄H₇₄N₄O₁₂ (M=851.08)

31.06 g (224.72 mmol) of potassium carbonate and 27.56 g (141.3 mmol) of bromoacetic acid-tert-butyl ester were added to a solution of 5.27 g (18.8 mmol) of 1e in 246 ml of acetonitrile-water mixture (5:1). The reaction suspension was heated to 70° C. and stirred for 24 hours. The suspension was concentrated by evaporation in a rotary evaporator, mixed with 350 ml of water and extracted three times with ethyl acetate. The organic phase was dried on sodium sulfate, filtered and evaporated to the dry state in a rotary evaporator. The crude product was purified by column chromatography (SiO₂, dichloromethane→dichloromethane:methanol 8:1). The desired product was produced with a yield of 71.0% (11.36 g, 13.35 mmol).

| | | | | |
|---|---|---|---|---|
| Cld.: | C 62.10 | H 8.76 | N 6.58 | O 22.56 |
| Fnd.: | C 61.98 | H 8.75 | N 6.57 | O 22.59 |

g) {[2-{[3-(Bis-carboxymethyl-amino)-pentyl]-carboxymethyl-amino}-1-(4-nitro-benzyl)-ethyl]-carboxymethyl-amino}-acetic acid

C₂₄H₃₄N₄O₁₂ (M=570.55)

20.18 g (23.71 mmol) of if was introduced into anisole. It was cooled to 0° C. 23 ml of trifluoroacetic acid was added. It was stirred at room temperature for 24 hours. The reaction solution was concentrated by evaporation. The residue was taken up in water and extracted three times with diethyl ether. The aqueous phase was mixed with 100 ml of 5% ammonia solution and freeze-dried. The operating step described most recently was repeated. The desired product was produced with a yield of 91% (12.31 g; 21.58 mmol).

| | | | | |
|---|---|---|---|---|
| Cld.: | C 50.52 | H 6.01 | N 9.82 | O 33.65 |
| Fnd.: | C 50.42 | H 6.00 | N 9.79 | O 33.69 |

### EXAMPLE 2

a) N³—[2-Amino-3-(4-nitro-phenyl)-propyl]-pentane-1,3-diamine

C₁₄H₂₄N₄O₂ (M=280.37)

2.92 g (7.67 mmol) of isolated by-products from 1d was dissolved in 40 ml of dichloromethane and mixed with 12.99 g (114.0 mmol) of trifluoroacetic acid. The solution was stirred for 90 minutes at room temperature and concentrated by evaporation in a rotary evaporator. It was mixed twice with dichloromethane and concentrated by evaporation again in each case. The crude product was mixed with 100 ml of 5% ammonia solution and freeze-dried. The operating step mentioned most recently was repeated. The desired product was produced with a yield of 87% (1.87 g, 6.67 mmol).

| | | | | |
|---|---|---|---|---|
| Cld.: | C 59.98 | H 8.63 | N 19.98 | O 11.41 |
| Fnd.: | C 59.89 | H 8.60 | N 19.93 | O 11.39 |

b) {[2-{[3-(Bis-tert-butoxycarbonylmethyl-amino)-1-ethyl-propyl]-tert-butoxycarbonylmethyl-amino}-1-(4-nitro-benzyl)-ethyl]-tert-butoxycarbonylmethyl-amino}-acetic acid tert-butyl ester

C₄₄H₇₄N₄O₁₂ (m=851.08)

11.80 g (85.4 mmol) of potassium carbonate and 10.47 g (53.7 mmol) of bromoacetic acid-tert-butyl ester were added to a solution of 6.18 g (7.14 mmol) of 2a in 246 ml of acetonitrile-water mixture (5:1). The reaction suspension was heated to 70° C. and stirred for 24 hours. The suspension was concentrated by evaporation in a rotary evaporator, mixed with 350 ml of water and extracted three times with ethyl acetate. The organic phase was dried on sodium sulfate, filtered off and evaporated to the dry state in a rotary evaporator. The crude product was purified by column chromatography (SiO₂, dichloromethane→dichloromethane:methane 8:1). The desired product was produced with a yield of 73.0% (4.44 g, 5.21 mmol).

| | | | | |
|---|---|---|---|---|
| Cld.: | C 62.10 | H 8.76 | N 6.58 | O 22.56 |
| Fnd.: | C 61.98 | H 8.77 | N 6.60 | O 22.55 |

c) {[2-{[3-Bis-carboxymethyl-amino)-1-ethyl-propyl]-carboxymethyl-amino}-1-(4-nitro-benzyl)-ethyl]-carboxymethyl-amino}-acetic acid

C₂₄H₃₄N₄O₁₂ (M=570.55)

1.35 g (1.58 mmol) of 2b was introduced into anisole. It was cooled to 0° C. 1.53 ml of trifluoroacetic acid was added. It was stirred at room temperature for 24 hours. The reaction solution was concentrated by evaporation. The residue was taken up in water and extracted three times with diethyl ether. The aqueous phase was concentrated by evaporation, mixed with 100 ml of 5% ammonia solution and freeze-dried. The operating step mentioned most recently was repeated. The desired product was produced with a yield of 88% (793 mg, 1.39 mmol).

| | | | | |
|---|---|---|---|---|
| Cld.: | C 50.52 | H 6.01 | N 9.82 | O 33.65 |
| Fnd.: | C 50.43 | H 6.02 | N 9.80 | O 33.61 |

### EXAMPLE 3

a) N-[2-(3-Amino-2,2-dimethyl-propylamino)-1-(4-nitro-benzyl)-ethyl]-2,2-dimethyl-propionamide

C₁₉H₃₂N₄O₃ (M=364.49)

13.8 g (134 mmol) of 1,3-diamino-2,2-dimethylpropane was added to a solution of 5.0 g (13.4 mmol) of mesylate from Example 1c in 5 ml of tetrahydrofuran and 2.1 ml (14.7 mmol) of triethylamine. The solution was heated for four hours to 50° C. The solution was concentrated by evaporation in a rotary evaporator. The residue was taken up in water and extracted three times with ethyl acetate. The organic phase was dried on sodium sulfate, filtered off, and concentrated by evaporation in a rotary evaporator. The residue was purified by column chromatography (SiO₂, dichloromethane→dichloromethane:methanol methanol:ammonia (10%) 10:1), yield 73.2% (3.58 g, 9.80 mmol).

| | | | | |
|---|---|---|---|---|
| Cld.: | C 62.61 | H 8.85 | N 15.37 | O 13.17 |
| Fnd.: | C 62.57 | H 8.86 | N 15.39 | O 13.17 |

b) N′-(3-Amino-2,2-dimethyl-propyl)-3-(4-nitro-phenyl)-propane-1,2-diamine

C₁₄H₂₄N₄O₂ (M=280.37)

5.23 g (13.75 mmol) of 3a was dissolved in 78 ml of dichloromethane and then mixed with 23.3 g (204.26 mmol) of trifluoroacetic acid. The solution was stirred for 90 minutes and concentrated by evaporation. The residue was taken up in 100 ml of 5% ammonia solution and freeze-dried. The operating step mentioned most recently was repeated. 8.21 g of product (13.2 mmol; 95.9%) was produced.

| | | | | |
|---|---|---|---|---|
| Cld.: | C 59.98 | H 8.63 | N 19.98 | O 11.41 |
| Fnd.: | C 59.91 | H 8.60 | N 19.91 | O 11.45 |

c) [(3-{[2-(Bis-tert-butoxycarbonylmethyl-amino)-3-(4-nitro-phenyl)-propyl]-tert-butoxycarbonylmethyl-amino}-2,2-dimethyl-propyl)-tert-butoxycarbonylmethyl-amino]-acetic acid tert-butyl ester

C₄₄H₂₄N₄O₁₂ (M=851.08)

8.02 g (12.9 mmol) of 3b was dissolved in 170 ml of an acetonitrile-water mixture (5:1) and mixed with 21.23 g (153.6 mmol) of potassium carbonate and 13.6 g (69.7 mmol) of bromoacetic acid-t-butyl ester. The reaction solution was heated at 70° C. for 24 hours. The suspension was concentrated by evaporation in a rotary evaporator. The residue was taken up in 300 ml of water and washed three times with ethyl acetate. The organic phase was dried with sodium sulfate, filtered off and evaporated to the dry state in a rotary evaporator. The residue was purified by column chromatography (SiO₂, hexane→ethyl acetate 1:1). 9.79 g (11.5 mmol; 89.32%) of the product that is mentioned in the title was produced.

| | | | | |
|---|---|---|---|---|
| Cld.: | C 62.10 | H 8.76 | N 6.58 | O 22.56 |
| Fnd.: | C 62.19 | H 8.74 | N 6.57 | O 22.59 |

d) [(3-{[2-(Bis-carboxymethyl-amino)-3-(4-nitro-phenyl)-propyl]-carboxymethyl-amino}-2,2-dimethyl-propyl)-carboxymethyl-amino]-acetic acid

C₂₄H₃₄N₄O₁₂ (M=570.55)

5.30 g (6.23 mmol) of 3c was introduced into 43 ml of anisole at −5° C. 6.15 ml (79.8 mmol) of trifluoroacetic acid was added. It was stirred overnight at room temperature. The reaction solution was concentrated by evaporation. The residue was taken up in water and extracted three times with diethyl ether. The aqueous phase was mixed with 100 ml of 5% ammonia solution and freeze-dried. 2.95 g (5.17 mmol) of the desired product was produced. This corresponds to a yield of 83%.

| | | | | |
|---|---|---|---|---|
| Cld.: | C 50.52 | H 6.01 | N 9.82 | O 33.65 |
| Fnd.: | C 50.43 | H 5.99 | N 9.85 | O 33.63 |

### EXAMPLE 4

a) 3-Hydroxy-2-methyl-propionamide

C₄H₉NO (M=103.12)

Analogously to: J. Amer. Chem. Soc.; 117; 9; (1995); 2479-2490. A solution that consists of 30.0 g (225 mmol) of β-hydroxy-isobutyric acid methyl ester and 750 ml of a 9M ammoniacal methanol solution was stirred in an airtight glass vessel for 7 days at 50° C. The solution was concentrated by evaporation in a vacuum. The residue was washed with cold diethyl ether (a total of 200 ml). A white solid of 15.77 g (153 mmol; 68%) remained.

| | | | | |
|---|---|---|---|---|
| Cld.: | C 46.59 | H 8.80 | N 13.58 | O 31.03 |
| Fnd.: | C 46.63 | H 8.83 | N 13.55 | O 31.06 |

b) 3-Amino-2-methyl-propan-1-ol

C₄H₁₁NO (M=89.14)

14.7 g (140 mmol) of 4a was suspended in 50 ml of tetrahydrofuran and mixed at 0° C. with 400 ml (400 mmol) of 1 M borane-THF-complex solution. The solution was refluxed for 4 hours and mixed at 0° C. with 70 ml of concentrated HCl solution. The solution was concentrated by evaporation in a rotary evaporator. Dilute sodium hydroxide solution (140 g of sodium hydroxide in 200 ml of water) was added at 0° C. to the solution. It was extracted four times with 100 ml each of chloroform. The combined organic phases were dried with magnesium sulfate, filtered and concentrated by evaporation. The residue was distilled in a water jet vacuum (92° C.). The desired product was produced with 9.23 g (103.6 mmol; 74%).

| | | | | |
|---|---|---|---|---|
| Cld.: | C 53.90 | H 12.44 | N 15.71 | O 17.95 |
| Fnd.: | C 53.80 | H 12.42 | N 15.68 | O 17.98 |

c) (3-Hydroxy-2-methyl-propyl)-carbamic acid tert-butyl ester

C₉H₁₉NO₃ (M=189.25)

63.0 g (333 mmol) of 4b was dissolved in 240 ml of tetrahydrofuran and cooled to 0° C. At this temperature, 72.6 g (329.5 mmol) of di-tert-butyldicarbonate ((Boc)₂O), dissolved in 95 ml of THF, was added in drops. It was heated to room temperature and stirred for one hour. The solution was concentrated by evaporation in a rotary evaporator. The residue was taken up in 400 ml of diethyl ether and washed with 100 ml of 0.01 N HCl, 100 ml of water and with 100 ml of sodium bicarbonate solution (5%). The organic phase was dried on sodium sulfate, filtered and concentrated by evaporation in a rotary evaporator.

| | | | | |
|---|---|---|---|---|
| Cld.: | C 57.12 | H 10.12 | N 7.40 | O 25.36 |
| Fnd.: | C 57.20 | H 10.10 | N 7.40 | O 25.39 |

d) Methanesulfonic acid 3-tert-butoxycarbonylamino-2-methyl-propyl ester

C₁₀H₂₁NO₅S (M=267.34)

29.7 ml (214.1 mmol) of triethylamine was added to 27.0 g (142.7 mmol) of 4c in 155 ml of dichloromethane. The solution was cooled to −5° C. and mixed with 11.7 ml (149.8 mmol) of methanesulfonic acid chloride, which had been dissolved ahead of time in 155 ml of dichloromethane. The suspension was stirred for two hours and mixed with 400 ml of water. The phases were separated. The aqueous phase was extracted twice with 150 ml each of dichloromethane. The combined, organic phases were washed twice with 200 ml of 0.1N HCl solution, once with 200 ml of 5% sodium bicarbonate solution and once with 100 ml of water. The organic phase was dried on sodium sulfate, filtered off and concentrated by evaporation in a rotary evaporator. The residue was recrystallized in 0° C. hexane. 34.7 g (129.9 mmol) of the desired product was produced; this corresponds to a yield of 91%.

| | | | | | |
|---|---|---|---|---|---|
| Cld.: | C 44.93 | H 7.92 | N 5.24 | O 29.92 | S 11.99 |
| Fnd.: | C 44.90 | H 7.89 | N 5.24 | O 29.90 | S 12.01 |

e) (3-Azido-2-methyl-propyl)-carbamic acid tert-butyl ester

C₉H₁₈N₄O₂ (M=214.27)

71.6 g (268.6 mmol) of 4d was dissolved in 490 ml of DMSO and mixed with 21.0 g (322.3 mmol) of sodium azide. The reaction solution was stirred for 24 hours at 40-45° C. The mixture was cooled to 25° C. and mixed with 500 ml of water. The solution was extracted five times with 250 ml of dichloromethane. The combined organic phases were washed twice with 150 ml each of saturated sodium chloride solution. The organic phase was dried with sodium sulfate, filtered off and concentrated by evaporation in a rotary evaporator. The residue was purified by column chromatography (SiO₂, hexane→hexane→ethyl acetate 1:1). After purification, 43.7 g (204 mmol) of product, which corresponds to a yield of 76%, was present.

| | | | | |
|---|---|---|---|---|
| Cld.: | C 50.45 | H 8.47 | N 26.15 | O 14.93 |
| Fnd.: | C 50.51 | H 8.26 | N 26.12 | O 14.95 |

f) (3-Amino-2-methyl-propyl)-carbamic acid tert-butyl ester

C₉H₂₀N₂O₂ (M=188.27)

30.8 g (143.8 mmol) of 4e was dissolved in 412 ml of ethyl acetate and mixed with 4.5 g of Pd/C (10%). The reaction solution was stirred at 25° C. under hydrogen atmosphere for 24 hours. The solution was filtered and concentrated by evaporation in a rotary evaporator. The residue was purified by column chromatography (SiO₂, dichloromethane→dichloromethane:methanol 1:1). Yield 24.28 g (129.0 mmol, 89.7%).

| | | | | |
|---|---|---|---|---|
| Cld.: | C 57.42 | H 10.71 | N 14.88 | O 17.00 |
| Fnd.: | C 57.45 | H 10.73 | N 14.90 | O 16.99 |

g) {3-[2-tert-Butoxycarbonylamino-3-(4-nitro-phenyl)-propionylamino]-2-methyl-propyl}-carbamic acid tert-butyl ester

C₂₃H₃₆N₄O₇ (M=480.56)

21.7 g (115 mmol) of 4f was dissolved in 600 ml of dichloromethane/water (1:1) and mixed with 36.0 g (115 mmol) of Boc-protected nitrophenylalanine and 17.6 g (115 mmol) of 1-hydroxybenzotriazole-H₂O (HOBT) The solution was cooled to about −5° C. 24.0 g (127 mmol) of 1-(dimethylaminopropyl)-3-ethylcarbodiimide (EDCI) was added and stirred for 7 hours and for another three days at 25° C. The phases were separated. The aqueous phase was extracted twice with dichloromethane. The combined, organic phases were washed twice with 150 ml each of saturated sodium bicarbonate solution and some water. The organic phase was dried with the sodium sulfate, filtered off and concentrated by evaporation in a rotary evaporator. The residue was pulverized in a mortar and washed with cold hexane. It was dried in an oil pump. Yield: 35.1 g (73.1 mmol, 63.6%).

| | | | | |
|---|---|---|---|---|
| Cld.: | C 57.49 | H 7.55 | N 11.66 | O 23.30 |
| Fnd.: | C 57.46 | H 7.55 | N 11.67 | O 23.32 |

h) 2-Amino-N-{3-amino-2-methyl-propyl}-3-(4-nitro-phenyl)-propionamide

C₁₃H₂₀N₄O₃ (M=280.33)

10.3 g (21.4 mmol) of 4g was suspended in 120 ml of dry dichloromethane. 24.5 ml (318 mmol) of trifluoroacetic acid was added in drops and stirred for one hour. The mixture was concentrated by evaporation in a rotary evaporator, mixed with 100 ml of dichloromethane and concentrated by evaporation again. The residue was washed with diethyl ether and dried at 40° C. in an oil pump. 100 ml of ammonia solution (5%) was added. It was freeze-dried. The desired product was produced with 5.55 g (19.80 mmol; 92.5%).

| | | | | |
|---|---|---|---|---|
| Cld.: | C 55.70 | H 7.19 | N 19.99 | O 17.12 |
| Fnd.: | C 55.39 | H 7.21 | N 19.89 | O 17.19 |

i) (3-Amino-2-methyl-propyl)-[2-amino-3-(4-nitro-phenyl)-propyl]-amine dihydrochloride

C₁₃H₂₂N₄O₂ (M=252.32)

9.25 g (18.2 mmol) of 4h was dissolved in 130 ml of absolute THF. 128.5 ml (128.5 mmol) of borane-THF complex (1 molar) was added in drops at 0° C. within 30 minutes. The solution was heated to room temperature and refluxed for 5 hours. The solution was cooled to 0° C., mixed with 35 ml of methanol, stirred for two hours and concentrated by evaporation. The residue was dissolved in 200 ml of ethanol, cooled in an ice bath and mixed with hydrogen chloride gas. The mixture was concentrated by evaporation and taken up in diethyl ether. The solid was suctioned off, flushed with diethyl ether and dried in a vacuum. 5.49 g (14.6 mmol: 80.3%) of the desired product was produced.

| | | | | | |
|---|---|---|---|---|---|
| Cld.: | C 41.56 | H 6.71 | N 14.91 | O 8.52 | Cl 28.31 |
| Fnd.: | C 41.59 | H 6.76 | N 14.92 | O 8.54 | Cl 28.26 |

k) [(3-{[2-(Bis-tert-butoxycarbonylmethyl-amino)-3-(4-nitro-phenyl)-propyl]-tert-butoxycarbonylmethyl-amino)-2-methyl-propyl)-tert-butoxycarbonylmethyl-amino]-acetic acid tert-butyl ester

C₄₃H₇₂N₄O₁₂ (M=837.06)

10.37 g (27.6 mmol) of 4i was dissolved in 300 ml of acetonitrile-water (5:1) and mixed with 45.5 g (329.2 mmol) of potassium carbonate. 29.1 g (149.4 mmol) of bromoacetic acid-tert-butyl ester was added. The batch was stirred at 70° C. for 24 hours. The reaction solution was mixed with 500 ml of water and extracted three times with 150 ml each of ethyl acetate. The organic phase was dried with sodium sulfate, filtered and concentrated by evaporation in a rotary evaporator. The residue was purified by column chromatography (SiO₂, dichloromethane→dichloromethane:methanol 8:1). The desired product was produced with a yield of 73.0% (16.87 g; 20.1 mmol).

| | | | | |
|---|---|---|---|---|
| Cld.: | C 61.70 | H 8.67 | N 6.69 | O 22.94 |
| Fnd.: | C 61.67 | H 8.66 | N 6.71 | O 22.91 |

l) [(3-{[2-(Bis-carboxymethyl-amino)-3-(4-nitro-phenyl)-propyl]-carboxymethyl-amino}-2-methyl-propyl)-carboxymethyl-amino]-acetic acid

C₂₃H₃₂N₄O₁₂=(M=556.52)

38.76 g (46.3 mmol) of 4k was introduced into 318 ml of anisole and cooled to −5C. 458 ml of trifluoroacetic acid was added. It was stirred for 3 hours at 0° C. It was heated to room temperature and stirred for 24 hours. The reaction solution was concentrated by evaporation in a rotary evaporator. The residue was taken up in 250 ml of water and extracted three times with diethyl ether. The aqueous phase was concentrated by evaporation. Methyl/ammonia (5%) were added and again concentrated by evaporation. Then, it was dissolved in water and freeze-dried. The desired product was produced with a yield of 98.3% (40.9 g; 45.5 mmol).

| | | | | |
|---|---|---|---|---|
| Cld.: | C 49.64 | H 5.82 | N 10.07 | O 34.50 |
| Fnd.: | C 49.53 | H 5.81 | N 10.01 | O 34.53 |

### EXAMPLE 5

a) 2-Benzyl-malonic acid diethyl ester

C₁₄H₁₆O₄ (M=250.29)

The synthesis of the desired product is known in the literature (Synthesis, 12, 2000, 1749-1755).

| | | | |
|---|---|---|---|
| Cld.: | C 67.18 | H 7.25 | O 25.57 |
| Fnd.: | C 67.15 | H 7.26 | O 25.54 |

b) 2-Benzyl-malonic acid amide

C₁₀H₁₂N₂O₂ (M=192.22)

The synthesis of the desired product has been performed analogously to Example 15b.

| | | | | |
|---|---|---|---|---|
| Cld.: | C 62.49 | H 6.29 | O 16.65 | N 14.57 |
| Fnd.: | C 62.59 | H 6.30 | O 16.64 | N 14.59 |

c) 2-Benzyl-propane-1,3-diamine

C₁₀H₁₆N₂ (M=164.25)

The reduction to the desired product has been performed analogously to Example 15c.

| | | | |
|---|---|---|---|
| Cld.: | 73.17 | H 9.82 | N 17.06 |
| Fnd.: | 73.09 | H 9.85 | N 17.09 |

d) [(2-Benzyl-3-{[2-(bis-carboxymethyl-amino)-3-(4-nitro-phenyl)-propyl]-carboxymethyl-amino}-propyl)-carboxymethyl-amino]-acetic acid

C₂₉H₃₆N₄O₁₂ (M=632.62)

The synthesis of the desired product and its precursors have been performed analogously to Example 15c.

| | | | | |
|---|---|---|---|---|
| Cld.: | C 55.06 | H 5.74 | N 8.86 | O 30.35 |
| Fnd.: | C 55.09 | H 5.72 | N 8.84 | O 30.32 |

### EXAMPLE 6

Gd Complex of the Compound According to Example 3

GdNa₂C₂₄H₂₉N₄O₁₂ (M=768.74)

142.6 mg (0.25 mmol) of 3d was suspended in 4 ml of distilled water, heated to 80° C. and brought into solution. It was mixed in portions with 45.3 mg (0.125 mmol) of Gd₂O₃. The suspension was heated to 80° C. and stirred for one hour. The solution was cooled to room temperature and set at pH=7 with sodium hydroxide solution (1 M). The water was removed by freeze-drying. The desired was produced with 192.2 mg (0.25 mmol, 99.8%).

| | | | | | | |
|---|---|---|---|---|---|---|
| Cld.: | C 37.50 | H 3.80 | N 7.29 | O 24.97 | Gd 13.87 | Na 5.98 |
| Fnd.: | C 37.49 | H 3.77 | N 7.31 | O 24.99 | Gd 13.89 | Na 6.01 |

### EXAMPLE 7

({3-[(2-(Bis-carboxymethyl-amino)-3-{4-[3-(2,5-dioxo-2,5-dihydro-pyrrol-1-yl)-propionylamino]-phenyl}-propyl)-carboxymethyl-amino]-2,2-dimethyl-propyl}-carboxymethyl-amino)-acetic acid

C₃₁H₄₁N₅O₁₃ (M=691.69)

270.3 mg (0.5 mmol) of aniline derivative 8 and 328 ml (4.54 mmol) of N-methylmorpholine were dissolved in 2.5 ml of dimethyl sulfoxide and mixed with 134.16 mg (0.61 mmol) of activated ester of maleimide, MPHS (Fluka). The reaction solution was heated, so that a homogeneous solution was produced. It was stirred for 40 minutes and concentrated by evaporation in a vacuum. The residue was purified by RP-HPLC. 180 mg (0.26 mmol; 52%) of the desired product was obtained.

| | | | | |
|---|---|---|---|---|
| Cld.: | C 53.83 | H 5.97 | N 10.13 | O 30.07 |
| Fnd.: | C 53.74 | H 6.00 | N 10.08 | O 30.09 |

### EXAMPLE 8

[(3-{[3-(4-Amino-phenyl)-2-(bis-carboxymethyl-amino)-propyl]-carboxymethyl-amino}-2,2-dimethyl-propyl}-carboxymethyl-amino]-acetic acid

C₂₄H₃₆N₄O₁₀ (M=540.57)

998 mg (1.75 mmol) of 3d and 0.6 g of palladium on carbon (10%) were dissolved in 30 ml of methanol-water (4:1) and hydrogenated under hydrogen atmosphere (normal pressure) at room temperature until the calculated amount of hydrogen (39.2 ml) had been taken up. It was filtered and rewashed with methanol. It was concentrated by evaporation in a rotary evaporator, suspended in toluene and concentrated by evaporation again. The desired product was produced with 746 mg (1.38 mmol; 78.7% yield).

| | | | | |
|---|---|---|---|---|
| Cld.: | C 53.33 | H 6.71 | N 10.36 | O 29.60 |
| Fnd.: | C 53.41 | H 6.74 | N 10.42 | O 29.61 |

### EXAMPLE 9

[(3-{[2-(Bis-carboxymethyl-amino)-3-(4-isothiocyanato-phenyl)-propyl]-carboxymethyl-amino}-2,2-dimethyl-propyl)-carboxymethyl-amino]-acetic acid

C₂₅H₃₄N₄O₁₀S (M=582.63)

811 mg (1.5 mmol) of product of Example 8 and 969 mg (9.14 mmol) of sodium carbonate were dissolved in 35 ml of distilled water and 70 ml of chloroform. 132.8 ml (1.74 mmol) of thiophosgene was added to this 2-phase system. The solution was stirred for 3 hours. The solution was concentrated by evaporation, taken up in 0.1 ml of dilute acetic acid (1%), and purified by means of RP-HPLC (25:74:1 acetonitrile/water/acetic acid). The desired product was produced with a yield of 64.2% (561 mg; 963 mmol).

| | | | | | |
|---|---|---|---|---|---|
| Cld.: | C 51.54 | H 5.88 | N 9.62 | O 27.46 | S 5.50 |
| Fnd.: | C 51.59 | H 5.88 | N 9.64 | O 27.50 | S 5.48 |

### EXAMPLE 10

{[3-({2-(Bis-carboxymethyl-amino)-3-[4-(2-bromo-acetylamino)-phenyl]-propyl}-carboxymethyl-amino)-2,2-dimethyl-propyl]-carboxymethyl-amino}-acetic acid

C₂₆H₃₇BrN₄O₁₁ (M=661.50)

81.1 mg (0.15 mmol) of product of Example 8 was dissolved in 3 ml of ethanol, 3 ml of distilled water and 3 ml of saturated sodium bicarbonate and mixed with 0.56 g (2.18 mmol) of bromoacetic acid anhydride. By the addition of solid sodium bicarbonate, the pH was kept at 8.5. The reaction solution was stirred for one hour and concentrated by evaporation. The residue was filtered over wadding, flushed with ethanol, concentrated by evaporation and purified by means of RP-HPLC. 79.4 mg (0.12 mmol) of product was produced. This corresponds to a yield of 80%.

| | | | | | |
|---|---|---|---|---|---|
| Cld.: | C 47.21 | H 5.64 | N 8.47 | O 26.60 | Br 12.08 |
| Fnd.: | C 47.11 | H 5.68 | N 8.49 | O 26.54 | Br 12.00 |

### EXAMPLE 11

{[3-({2-(Bis-carboxymethyl-amino)-3-[4-(2-iodo-acetylamino)-phenyl]-propyl}-carboxymethyl-amino)-2,2-dimethyl-propyl]-carboxymethyl-amino}-acetic acid

C₂₆H₃₇IN₄O₁₁ (M=708.50)

81.1 mg (0.15 mmol) of product of Example 8 was dissolved in 3 ml of ethanol, 3 ml of distilled water and 3 ml of saturated sodium bicarbonate and mixed with 771 mg (2.18 mmol) of iodoacetic acid anhydride. By the addition of solid sodium bicarbonate, the pH was kept at 8.5. The reaction solution was stirred for one hour and concentrated by evaporation. The residue was filtered over wadding, flushed with ethanol, concentrated by evaporation and purified by means of RP-HPLC. A yield of 69% (73.3 mg; 0.104 mmol) of the desired product was produced.

| | | | | | |
|---|---|---|---|---|---|
| Cld.: | C 44.08 | H 5.26 | N 7.91 | O 24.84 | I 17.91 |
| Fnd.: | C 44.04 | H 5.29 | N 7.89 | O 24.75 | I 17.99 |

### EXAMPLE 12

[(3-{[2-(Bis-carboxymethyl-amino)-3-(4-thiosemicarbazido-phenyl)-propyl]-carboxymethyl-amino}-2,2-dimethyl-propyl)-carboxymethyl-amino]-acetic acid

C₂₅H₃₈N₆O₁₀S (M=614.67)

The desired product was obtained from the compound of Example 8, analogously to the instructions in Collect. Czech. Chem. Commun., 57, 3, (1992), 656-659.

| | | | | | |
|---|---|---|---|---|---|
| Cld.: | C 48.85; | H 6.23; | N 13.67; | O 26.03; | S 5.22 |
| Fnd.: | C 48.77; | H 6.25; | N 13.62; | O 26.06; | S 5.25 |

### EXAMPLE 13

[(3-{[3-(4-Acetylamino-phenyl)-2-(bis-carboxymethyl-amino)-propyl]-carboxymethyl-amino}-2,2-dimethyl-propyl)-carboxymethyl-amino]-acetic acid

C₂₆H₃₈N₄O₁₁ (M=582.60)

Analogously to J. Amer. Chem. Soc., 120; 12; 1998; 2768-2779:

81.1 mg (0.15 mmol) of product of Example 8 was dissolved in 4.5 ml of acetonitrile-H₂O (9:1), cooled to 0° C. and mixed with 38.3 mg (375 mmol) of acetic acid anhydride. The solution was stirred for 4 hours at room temperature. It was filtered and concentrated by evaporation in a vacuum.

| | | | | |
|---|---|---|---|---|
| Cld.: | C 53.60 | H 6.57 | N 9.62 | O 30.21 |
| Fnd.: | C 53.49 | H 6.54 | N 9.64 | O 30.24 |

### EXAMPLE 14

a) 2,3-Dimethyl-succinonitrile

C₆H₈N₂ (M=108.14)

The synthesis of 2,3-dimethyl-succinonitrile was performed according to a procedure by Whiteley and Marianelli (Synthesis (1978), 392-394) from 8.7 g (149.8 mmol) of acetone, 16 ml (150.4 mmol) of ethyl cyanoacetate and 10 g (154 mmol) of potassium cyanide. The crude product was purified by distillation in a vacuum. Yield: 7.0 g (64.7 mmol; 61%).

| | | | |
|---|---|---|---|
| Cld.: | C 66.64 | H 7.46 | N 25.90 |
| Fnd.: | C 66.60 | H 7.44 | N 25.92 |

b) 2,3-Dimethyl-butane-1,4-diamine

C₈H₁₅N₂ (M=116.21)

The synthesis of 2,3-dimethyl-butane-1,4-diamine was performed according to instructions from Alzencang et al. (J. Med. Chem. 38; 21; 1995; 4337-4341): 10.81 mg (100 mmol) of 2,3-dimethyl-succinonitrile was reacted with saturated diborane-THF solution. 8.02 g (69 mmol; 69%) of desired product was produced as a colorless liquid.

| | | |
|---|---|---|
| C 62.02 | H 13.88 | N 24.11 |
| C 62.19 | H 13.90 | N 24.12 |

c) [(4-{[2-(Bis-carboxymethyl-amino)-3-(4-nitro-phenyl)-propyl]-carboxymethyl-amino}-2,3-dimethyl-butyl}-carboxymethyl-amino]-acetic acid

C₂₅H₃₆N₄O₁₂ (M=584.58)

The synthesis of the desired product and the corresponding precursors was performed analogously to Example 3.

| | | | | |
|---|---|---|---|---|
| Cld.: | C 51.37 | H 6.21 | N 9.58 | O 32.84 |
| Fnd.: | C 51.34 | H 6.23 | N 9.61 | O 32.80 |

### EXAMPLE 15

a) Cyclopentane-1,1-dicarboxylic acid diethyl ester

C₁₁H₁₈O₄ (M=214.26)

The synthesis of the substance was performed from 1,4-dibromobutane and malonic acid diester according to instructions from J. Amer. Chem. Soc., 109; 22; 1987; 6825-6836.

| | | | |
|---|---|---|---|
| Cld.: | C 61.66 | H 8.47 | O 29.87 |
| Fnd.: | C 61.69 | H 8.46 | O 29.82 |

b) Cyclopentane-1,1-dicarboxylic acid diamide

C₇H₁₂N₂O₂ (M=156.18)

A solution of 21.4 g (100 mmol) of 15a and 500 ml of a 9 M ammoniacal methanol solution was stirred in an airtight glass vessel for 7 days at 50° C. The solution was concentrated by evaporation in a vacuum. The residue was washed with cold diethyl ether (a total of 200 ml). A white solid of 10.0 g (64 mmol; 64%) remained.

| | | | | |
|---|---|---|---|---|
| Cld.: | C 53.83 | H 7.74 | N 17.97 | O 20.49 |
| Fnd.: | C 53.80 | H 7.71 | N 18.00 | O 20.52 |

c) C—(1-Aminomethyl-cyclopentyl)-methylamine

C₇H₁₆N₂ (M=128.22)

9.9 g (63.4 mmol) of 15b was suspended in 50 ml of tetrahydrofuran. 400 ml (400 mmol) of 1 M-borane-THF complex solution was added at 0° C. The solution was refluxed for 4 hours and mixed at 0° C. with 70 ml of concentrated HCl solution. The solution was concentrated by evaporation in a rotary evaporator. Dilute sodium hydroxide solution (140 g of sodium hydroxide in 200 ml of water) was added at 0° C. to the solution. It was extracted four times with 100 ml each of chloroform. The combined, organic phases were dried with magnesium sulfate, filtered and concentrated by evaporation. The residue was distilled in a vacuum. The desired product was produced with 5.85 g (45.64 mmol; 72%).

| | | | |
|---|---|---|---|
| Cld.: | C 65.57 | H 12.58 | N 21.85 |
| Fnd.: | C 65.49 | H 12.59 | N 21.87 |

d) {[1-({[2-(Bis-carboxymethyl-amino)-3-(4-nitro-phenyl)-propyl]-carboxymethyl-amino}-methyl)-cyclopentylmethyl]-carboxymethyl-amino}-acetic acid

C₂₆H₃₆N₄O₁₂ (M=596.59)

The synthesis of the desired product and the corresponding precursors was performed analogously to Example 3 with the diamine 15c.

| | | | | |
|---|---|---|---|---|
| Cld.: | C 52.35 | H 6.08 | N 9.39 | O 32.18 |
| Fnd.: | C 52.43 | H 6.09 | N 9.40 | O 32.21 |

### EXAMPLE 16

trans-[(4-{[2-(Bis-carboxymethyl-amino)-3-(4-nitro-phenyl)-propyl]-carboxymethyl-amino)-cyclohexyl)-carboxymethyl-amino]-acetic acid

C₂₅H₃₄N₄O₁₂ (M=584.58)

The synthesis of the desired product and the corresponding precursors was performed analogously to Example 3 with trans-1,4-diaminocyclohexane.

| | | | | |
|---|---|---|---|---|
| Cld.: | C 51.37 | H 6.21 | N 9.58 | O 32.84 |
| Fnd.: | C 51.28 | H 6.23 | N 9.60 | O 32.88 |

### EXAMPLE 17

a) cis-2-Aminomethyl-cyclohexylamine

C₆H₁₄N₂ (M=114.19)

The compound has been produced as described in the literature (Tetrahedron; 44; 5; 1988; 1465-1476).

| | | | |
|---|---|---|---|
| Cld.: | C 65.57 | H 12.58 | N 21.85 |
| Fnd.: | C 65.51 | H 12.61 | N 21.87 |

b) {[2-{[2-(Bis-carboxymethyl-amino)-cyclohexylmethyl]-carboxymethyl-amino}-1-(4-nitro-benzyl)-ethyl]-carboxymethyl-amino}-acetic acid

C₂₆H₃₆N₄O₁₂ (M=596.59)

The synthesis of the desired product and the corresponding precursors was performed analogously to Example 3 with the diamine 17a.

| | | | | |
|---|---|---|---|---|
| Cld.: | C 52.35 | H 6.08 | N 9.39 | O 32.18 |
| Fnd.: | C 52.30 | H 6.09 | N 9.36 | O 32.20 |

### EXAMPLE 18

Antibody Conjugate of {[3-({2-(Bis-carboxymethyl-amino)-3-[4-(2-bromo-acetylamino)-phenyl]-propyl}-carboxymethyl-amino)-2,2-dimethyl-propyl]-carboxymethyl-amino}-acetic acid

200 μg of an antibody with freely accessible thiol groups (e.g., HuM195 (cf. Michael R. McDevitt, J. Nuc. Med. 40, 1999, 1772; commercially available from Protein Design Labs Inc., Mountainview, Calif., US)—if the antibody does not have any freely accessible thiol groups, the latter can be produced by the use of 2-iminothiolane HCl (e.g., EP 0 607 222 B1)) was diluted in 1.2 ml of borate buffer (50 mmol, pH 8.5), mixed with 238 μg (240 nmol) of product of Example 10, dissolved in 50 μl of borate buffer (see above), and stirred for 3 hours at 37° C. It was purified on a NAP-5 column (Amersham Pharmacia Biotech AB, Sephadex G-25, Mobile Phase: PBS).

### EXAMPLE 19

Indium 111-Labeled Antibody Conjugate of {[3-({2-(Bis-carboxymethyl-amino)-3-[4-(2-bromo-acetylamino)-phenyl]-propyl]-carboxymethyl-amino)-2,2-dimethyl-propyl]-carboxymethyl-amino}-acetic acid

200 μg of an antibody with freely accessible thiol groups (e.g., HuM195 (cf. Michael R. McDevitt, J. Nuc. Med. 40, 1999, 1722; commercially available from Protein Design Labs Inc., Mountainview, Calif., USA)—if the antibody does not have any freely accessible thiol groups, the latter can be produced by the use of 2-iminothiolane HCl (e.g., EP 0 607 222 B1)) was diluted in 1.2 ml of borate buffer (50 mmol, pH 8.5), mixed with 238 μg (240 nmol) of product of Example 10, dissolved in 50 μl of borate buffer (see above), and stirred for 3 hours at 37° C. The borate buffer solution was exchanged for an acetate buffer, by the sample solution being set at 0.1 M (pH 6.0) three times for 1 hour in the Slide-A-Lyzer 10000, Pierce MWCO (dialysis process) against 200 ml of NaOAc buffer in each case. Finally, it was set at 0.1 M (pH 6) overnight against 400 ml of NaOAc buffer. The solution was mixed with 80 μl (0.05 M HCl) of [¹¹¹In]InCl₃ (27.88 MBq) and stirred for 30 minutes at room temperature. It was purified on an NAP-5 column (Amersham Pharmacia Biotech AB, Sephadex G-25, Mobile Phase: PBS).

### EXAMPLE 20

Yttrium 90-Labeled Antibody Conjugate of {[3-({2-(Bis-carboxymethyl-amino)-3-[4-(2-bromo-acetylamino)-phenyl]-propyl}-carboxymethyl-amino)-2,2-dimethyl-propyl]-carboxymethyl-amino}-acetic acid

200 μg of an antibody with freely accessible thiol groups (e.g., HuM195 (cf. Michael R. McDevitt, J. Nuc. Med. 40, 1999, 1722; commercially available from Protein Design Labs Inc., Mountainview, Calif., USA)—if the antibody does not have any freely accessible thiol groups, the latter can be produced by the use of 2-iminothiolane HCl (e.g., EP 0 607 222 B1)) was diluted in 1.2 ml of borate buffer (50 mmol, pH 8.5), mixed with 238 μg (240 nmol) of product from Example 10, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaoAc buffer. The solution was mixed with 50 MBq of [⁹⁰Y]YCl₃ and stirred for 30 minutes at room temperature. It was purified on an NAP-5 column (Amersham Pharmacia Biotech AB, Sephadex G-25, Mobile Phase: PBS).

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of formula I 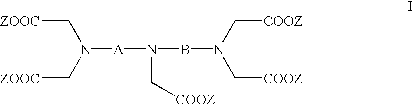
in which
Z stands for a hydrogen atom or a metal ion equivalent,
A stands for a radical of formula 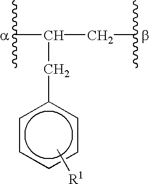
in which positions α and β that are characterized by are bonded to any of the adjacent nitrogen atoms, R¹ is a nitro group or a group that can enter into a reaction with a biomolecule, and
B stands for a radical of formula 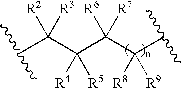
in which n is 0 or 1, and R², R³, R⁴, R⁵₁ R⁶₁ R⁷₁ R⁸ and R⁹, independently of one another, are selected from a hydrogen atom, a straight-chain or branched, saturated or unsaturated C₁₋₆ alkyl group, which optionally can be substituted with 1 or 2 hydroxy groups and/or can contain 1 or 2 oxygen atoms, and an aralkyl group, whose aryl radical optionally can be substituted with an alkyl or alkoxy group, whereby two of radicals R², R³, R⁴, R⁵, R⁶, R⁷, R⁸ and R⁹ can be part of a 5- or 6-membered ring, provided that at least one and at most four of radicals R², R³, R⁴, R⁵, R⁶, R⁷, R⁸, and R⁹ are not hydrogen atoms, as well as salts thereof.

2. Compounds according to claim 1, in which radical A is bonded via the α-position to the (ZOOC—CH₂)₂—N radical.

3. Compounds according to one of the preceding claims, in which R¹ is selected from the group that consists of nitro, amino, isocyanate, isothiocyanate, hydrazine, semicarbazide, thiosemicarbazide, chloroacetamide, bromoacetamide, iodoacetamide, acylamino, maleimide, maleimidacylamino, activated esters, mixed anhydrides, azide, hydroxide, sulfonyl chloride and carbodiimide.

4. Compounds according to one of the preceding claims, in which 1 or 2 of the radicals R², R³, R⁴, R⁵, R6, R⁷, R⁸ and R⁹ are selected from the group that consists of methyl, ethyl and benzyl, and the others of these radicals are hydrogen atoms.

5. Compounds according to one of the preceding claims, in which radicals R², R³, R⁴, R⁵, R⁶, R⁷, R⁸ and R⁹ are selected such that B is symmetrical.

6. Compounds according to one of claims 1-3, in which B is selected from the group that consists of —CH₂—CH₂—CH(CH₂—CH₃)—, —CH(CH₂—CH₃)—CH₂—CH₂—, —CH₂—C(CH₃)₂—CH₂—, —CH₂—CH(CH₃)—CH₂—, —CH₂—CH(CH₂-phenyl)—CH₂—, —CH₂—CH(CH₃)—CH(CH₃)—CH₂—,

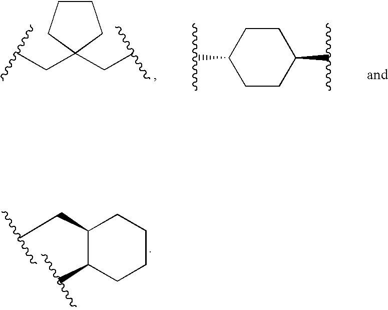

7. Compounds according to one of the preceding claims, in which n=0.

8. Compounds according to one of the preceding claims, in which at least two of radicals Z stand for a metal ion equivalent of a paramagnetic element of atomic numbers 21-29, 42, 44 and 58-70.

9. Compounds according to one of claims 1-7, in which at least two of radicals Z stand for a metal ion equivalent of a radioactive element of atomic numbers 26, 27, 29, 31, 32, 37-39, 43, 46, 47, 49, 61, 62, 64, 67, 70, 71, 75, 77, 82 and 83.

10. Conjugates of general formula II 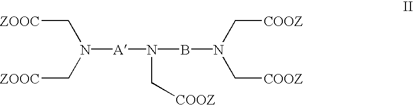
in which Z and B are defined as in claim 1, and A′ stands for a radical of formula 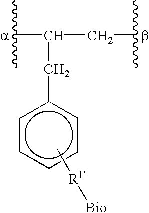
in which positions α and β that are characterized by are bonded to any of the adjacent nitrogen atoms, and Bio stands for the radical of a biomolecule, which is bonded via radical R¹′ of a reactive group to the phenylene ring, as well as salts thereof.

11. Conjugates according to claim 10, in which the biomolecule is selected from the group that consists of biopolymers, proteins, synthetically modified biopolymers, carbohydrates, antibodies, DNA and RNA fragments, β-amino acids, vector amines for transfer into the cell, biogenic amines, pharmaceutical agents, oncological preparations, synthetic polymers, which are directed to a biological target, steroids, prostaglandins, taxol and derivatives thereof, endothelins, alkaloids, folic acid and derivatives thereof, bioactive lipids, fats, fatty acid esters, synthetically modified mono-, di- and tri-glycerides, liposomes that are derivatized on the surface, micelles that consist of natural fatty acids or perfluoroalkyl compounds, porphyrins, texaphrines, expanded porphyrins, cytochromes, inhibitors, neuramidases, neuropeptides, immunomodulators, endoglycosidases, substrates that are attacked by the enzymes calmodolin kinase, casein-kinase II, glutathione-S-transferase, heparinase, matrix-metalloproteases, β-insulin-receptor-kinase, UDP-galactose, 4-epimerase, fucosidases, G-proteins, galactosidases, glycosidases, glycosyl transferases and xylosidases; antibiotics, vitamins and vitamin analogs, hormones, DNA-intercalators, nucleosides, nucleotides, lectins, vitamin B12, Lewis-X and related substances, psoralens, dienetriene antibiotics, carbacyclins, VEGF, somatostatin and derivatives thereof, biotin derivatives, antihormones, tumor-specific proteins and synthetic agents, dendrimers and cascade polymers, as well as derivatives thereof, polymers that accumulate in acidic or basic areas of the body, myoglobins, apomyoglobins, neurotransmitter peptides, tumor necrosis factors, peptides that accumulate in inflamed tissues, blood-pool reagents, anion and cation-transporter proteins, polyesters, polyamides and polyphosphates.

12. Conjugates according to one of claims 10 or 11, in which at least two of radicals Z stand for a metal ion equivalent of a paramagnetic element of atomic numbers 21-29, 42, 44 and 58-70.

13. Conjugates according to one of claims 10 or 11, in which at least two of radicals Z stand for a metal ion equivalent of a radioactive element of atomic numbers 26, 27, 29, 31, 32, 37-39, 43, 46, 47, 49, 61, 62, 64, 67, 70, 71, 75, 77, 82 and 83.

14. Use of a compound according to one of claims 1-9 for the production of a conjugate with a biomolecule.

15. Pharmaceutical agents that contains at least one physiologically compatible compound according to one of claims 8 or 9 or at least one physiologically compatible conjugate according to one of claims 12 or 13, optionally with the additives that are commonly used in galenicals.

16. Use of a compound according to one of claims 1-8 or a conjugate according to one of claims 10-12 for the production of agents for NMR diagnosis.

17. Use of a compound according to one of claims 1-7 or 9 or a conjugate according to one of claims 10, 11 or 13 for the production of agents for radiodiagnosis or radiotherapy.

18. Kit for the production of radiopharmaceutical agents, comprising a compound according to one of claims 1-7 or a conjugate according to one of claims 10 or 11, in which Z is hydrogen, and a compound of a radioactive element of atomic numbers 26, 27, 29, 31, 32, 37-39, 43, 46, 47, 49, 61, 62, 64, 67, 70, 71, 75, 77, 82 and 83.

19. Process for the production of a compound according to claim 1 or a conjugate according to claim 10, in which a compound of formula III
H₂N—A—NH—B—NH₂ III
whereby A and B are as defined in claim 1, is reacted with a compound of formula IV
Nu—CH₂—COOZ′ IV,
whereby Nu stands for a nucleofuge and Z′ stands for a hydrogen atom, a metal ion equivalent or a protective group for carboxyl, then the compound that is thus obtained is optionally reacted with a biomolecule, whereby the radical R¹, if it is nitro, first must be converted into a group that can enter into a reaction with a biomolecule and after that, and after the removal of optionally still present protective groups, and in a way that is known in the art, is reacted, if desired, with at least one metal oxide or metal salt and optionally then acidic hydrogen atoms that are still present in the complexes that are thus obtained are substituted completely or partially by cations of inorganic and/or organic bases, amino acids or amino acid amides.
